(19) United States
(12) Patent Application Publication
(10) Pub. No.: US 2021/0213447 A1
(43) Pub. Date: Jul. 15, 2021

(54) PATTERNED FLOW CELLS FOR BIOMOLECULAR ANALYSIS

(71) Applicant: CORNING INCORPORATED, CORNING, NY (US)

(72) Inventor: Ye Fang, Painted Post, NY (US)

(21) Appl. No.: 17/251,011

(22) PCT Filed: Jun. 10, 2019
(86) PCT No.: PCT/US2019/036309

§ 371 Date: Dec. 10, 2020

Related U.S. Application Data
(60) Provisional application No. 62/685,091, filed on Jun. 14, 2018.

(51) Int. Cl. B01L 3/00

(52) U.S. Cl. CPC B01L 3/502707; B01L 2200/0668; B01L 2300/0819; B01L 2300/123; B01L 2300/0896; B01L 2300/16; B01L 2300/0877

(57) ABSTRACT

A patterned flow cell includes a substrate (100, 200) having a patterned array of metal oxide nano-patches (104, 202). Each of the metal oxide nano-patches (104, 202) has an organophosphate coating layer (106, 206) to increase the ability of the metal oxide (104, 204) to bind with DNA, proteins, or polynucleotides. A silane coating layer (108, 208) is deposited in the interstitial spaces on the substrate (100, 200) between the metal oxide nano-patches (104, 202) to prevent the binding of polynucleotides, DNA, or proteins in the interstitial spaces.

### CROSS-REFERENCE TO RELATED APPLICATIONS

This application claims the benefit of priority under 35 U.S.C. § 119 of U.S. Provisional Application No. 62/685,091, filed Jun. 14, 2018, the content of which is incorporated herein by reference in its entirety.

### FIELD

This present disclosure generally relates to biomolecular analysis, and more specifically to methods and microfluidic flow cell devices for massively parallel genomics analysis.

### BACKGROUND

Biological samples are often complicated in composition and amount. Analysis of biomolecules in a biological sample often involves the partition of the single sample into tens of thousands or millions of samples for quantitative determination. One of the popular means to achieve this is to use a solid substrate surface to selectively immobilize and partition different biomolecules in the biological sample.

The past decades have witnessed extraordinary progress in cataloguing human genetic variations, correlating these variations with susceptibility to disease, responsiveness to specific therapies, and susceptibility to dangerous drug side effects and other medically actionable characteristics. Massively parallel gene sequencing (also termed next-generation sequencing (NGS)) is a very effective means to perform this gene testing. Recent advances in genome sequencing technologies have led to a decreased cost per megabase and an increase in the number and diversity of genomes sequenced.

Embodiments in the present disclosure address some of the aforementioned shortcomings associated with DNA sequencing, and provide an improvement over the state of the art with respect to genome-wide sequencing. These and other advantages, as well as additional inventive features, will be apparent from the description provided herein.

### SUMMARY

Embodiments in the present disclosure include a patterned surface having an array of metal oxide patches or islands, where, in some embodiments, the metal oxide patches have an organophosphate coating that supports the binding of DNA nanoballs or a DNA-primer-containing polymer. The interstitial region between adjacent metal oxide patches is optionally coated with a silane that is resistant to the binding of nucleotides, DNA, or proteins. Also provided are methods for making such selective coating surfaces. Certain embodiments also provide a method for making an array of nucleic acids or other analytes using such selective coating surfaces.

In some embodiments, a patterned flow cell includes a substrate having a patterned array of metal oxide nano-patches. Each of the metal oxide nano-patches has an organophosphate coating layer configured to increase the ability of the metal oxide to bind with DNA, proteins, and/or polynucleotides. A silane coating layer is deposited in the interstitial spaces on the substrate between the metal oxide nano-patches. The silane coating is configured to prevent the binding of polynucleotides, DNA, and/or proteins in the interstitial spaces.

In various embodiments, the metal oxide is one of Al₂O₃, ZnO₂, Ta₂O₅, Nb₂O₅, SnO₂, In₂O₃, NiO, a-TiO₂, r-TiO₂, MgO, indium tin oxide, indium zinc oxide, CeO₂, CoO, Co₃O₄, CrO₃, Fe₂O₃, Fe₃O₄, WO₃, Y₂O₃, ZrO₂, and Mn₂O₃. In some embodiments, the shape of the nano-patch is one of rectangular, circular, elliptical, oval, triangular, and trapezoidal, and/or the thickness of the metal oxide is in a range from one nanometer to five micrometers (μm). The substrate may be made from glass, quartz, silicon, or plastic.

In some embodiments, the silane coating layer includes polyethylene-glycol-containing silane, while the organophosphate is one of amine-terminated organophosphate, epoxy-containing organophosphate, and carboxylate organophosphate. In some embodiments, the metal oxide is transparent to light with wavelengths in a range from 400 nanometers (nm) to 700 nanometers (nm).

In some embodiments, a patterned flow cell includes a substrate having a patterned array of silicon dioxide (SiO₂) or silicate nano-patches. Each of the SiO₂ or silicate nano-patches has a silane coating layer configured to increase the ability of the SiO₂ or silicate to bind with DNA, proteins, and/or polynucleotides. An organophosphate coating layer can be deposited in the interstitial metal oxide region on the substrate between the SiO₂/silicate nano-patches. The organophosphate coating can be configured to prevent the binding of polynucleotides, DNA, and/or proteins in the interstitial spaces. Silicon can have a layer of SiO₂ once exposed to air, or can thermally grow a layer of SiO₂, when quartz or pure silica substrate surface contains SiO₂, and glass is a silicate substrate.

In some embodiments, the metal oxide is one of Al₂O₃, ZnO₂, Ta₂O₅, Nb₂O₅, SnO₂, In₂O₃, NiO, a-TiO₂, r-TiO₂, MgO, indium tin oxide, indium zinc oxide, CeO₂, CoO, Co₃O₄, CrO₃, Fe₂O₃, Fe₃O₄, WO₃, Y₂O₃, ZrO₂, and Mn₂O₃. In some embodiments, the shape of the nano-patch is one of rectangular, circular, elliptical, oval, triangular, and trapezoidal, and/or the thickness of the metal oxide is in a range from one nanometer to five micrometers. The substrate may be made from glass, quartz, pure silica, or silicon.

In some embodiments, the organophosphate is one of a polyethylene glycol-containing organophosphate and polyvinyl phosphoric acid, and/or the silane coating layer is one of amine-terminated silane, epoxy-containing silane, maleimide polyethylene-glycol-containing silane, and carboxylate silane.

In some embodiments, a method of manufacturing a patterned flow cell includes the steps of depositing an array of metal oxide nano-patches onto a substrate, coating the metal oxide nano-patches with a first material which increases the ability of the metal oxide to bind with DNA, proteins, and/or polynucleotides, and depositing a coating layer of a second material on the substrate in the interstitial spaces between the nano-patches. The second material can be configured to prevent the binding of polynucleotides, DNA, and/or proteins in the interstitial spaces.

In some embodiments, the method includes depositing the array of metal oxide nano-patches using a combination of photolithography and metal-oxide deposition. In some embodiments, the first material is one of amine-terminated organophosphate, epoxy-containing organophosphate, and carboxylate organophosphate or, alternatively, one of amine-terminated silane, epoxy-containing silane, maleimide polyethylene-glycol-containing silane, and carboxylate silane. The first material may be deposited as a solution-based coating or as a vapor-based coating depending on the nature of the first materials. For instance, silane coating can be achieved via a vapor-based coating approach, while organophosphate coating can be achieved via a solution based coating approach. In some embodiments, the second material is a polyethylene-glycol-containing silane or, alternatively, one of a polyethylene glycol-containing organophosphate and polyvinyl phosphoric acid. The second material may be deposited as a solution-based coating or as a vapor-based coating.

In some embodiments, a method of manufacturing a patterned flow cell includes the steps of forming an array of SiO₂ or silicate nano-patches onto a metal-oxide-coated substrate, depositing a coating layer of a first material on the substrate in the interstitial spaces between the nano-patches, the first material configured to prevent the binding of polynucleotides, DNA, and/or proteins in the interstitial spaces, and coating the SiO₂ or silicate nano-patches with a second material which increases the ability of the SiO₂ or silicate to bind with DNA, proteins, and/or polynucleotides.

In some embodiments, forming the array of SiO₂ or silicate nano-patches comprises depositing a metal oxide layer on a substrate, forming patterns using photolithography, and removing portions of the metal oxide layer within the exposed pattern region using reactive ion etching. The first material may be polyethylene glycol-containing organophosphate or polyvinyl phosphoric acid. The second material may be one of amine-terminated silane, epoxy-containing silane, maleimide polyethylene-glycol-containing silane, and carboxylate silane.

Additional features and advantages will be set forth in the detailed description which follows, and in part will be readily apparent to those skilled in the art from that description or recognized by practicing the embodiments as described herein, including the detailed description which follows, the claims, as well as the appended drawings.

It is to be understood that both the foregoing general description and the following detailed description are merely exemplary, and are intended to provide an overview or framework to understanding the nature and character of the claims. The accompanying drawings are included to provide a further understanding, and are incorporated in and constitute a part of this specification. The drawings illustrate one or more embodiment(s), and together with the description serve to explain principles and operation of the various embodiments.

### BRIEF DESCRIPTION OF THE DRAWINGS

The accompanying drawings incorporated in and forming a part of the specification illustrate several aspects of the present disclosure. In the drawings:

FIG. 1 is a diagrammatic representation of a method for making a microfluidic flow cell device including a substrate having selective patterned surfaces using a combination of lift-off photolithography, metal oxide layer deposition, and organophosphate coating, and glass-to-glass bonding, in accordance with exemplary embodiments;

FIG. 2 is a diagrammatic representation of a method for making a microfluidic flow cell device including a substrate having selective patterned surfaces using a combination of lift-off photolithography, metal oxide layer deposition, organophosphate and silane coating, and glass-to-glass bonding, in accordance with exemplary embodiments;

FIGS. 3A, 3B, 3C and 3D are diagrammatic representations of different variations of selective patterned surfaces that can be achieved by combining organophosphate with silane chemistry, according to exemplary embodiments.

While various embodiments will be disclosed hereinbelow, there is no intent to be limited to those embodiments. On the contrary, the intent is to cover all alternatives, modifications and equivalents as included within the spirit and scope of the disclosure as defined by the appended claims.

### DETAILED DESCRIPTION

Applicants have discovered that advances in genome-wide sequencing may be enabled by a microfluidic flow cell to partition a genomic DNA sample containing millions of DNA fragments onto the inner surface of the flow cells so almost all fragments immobilized can be sequenced simultaneously. Embodiments presented herein disclose microfluidic flow cell devices that can be used as a convenient format for housing an array of nucleic acids and that is subjected to sequencing-by-synthesis (SBS) or sequencing-by-ligation (SBL) or other detection technique that involves repeated delivery of reagents in cycles.

Over the past decade, NGS technologies have continued increasing capacity and throughput and reducing the cost of sequencing a genome. Conventional NGS platforms can provide vast quantities of genetic data, but the associated error rates are higher and the read lengths are generally shorter than those of traditional Sanger sequencing platforms. Thus, continued innovations in sequencing methodologies and the systems used to carry them out are required, and are disclosed herein.

In the sequencing-by-synthesis technique using cyclic reversible termination approaches, this NGS technique can use patterned nano-well flow cells, where a single DNA fragment is captured and located inside a nano-well and amplified up to 1,000 times to form a monoclonal cluster of the DNA fragment. The cluster generation can be achieved using exclusion amplification approach. Afterwards, the sequence of the DNA fragment at each well can be determined by synthesis. Here, a mixture of primers, DNA polymerase and modified nucleotides can be added to the flow cell. Each nucleotide can be blocked by a 3′-O-azidomethyl group and labeled with a nucleotide-specific, cleavable fluorophore. During each cycle, fragments in each cluster can incorporate just one nucleotide as the blocked 3′ group prevents additional incorporations.

After nucleotide incorporation, unincorporated bases can be washed away, and the surface can be imaged by fluorescence microscopy using either two or four laser channels; the color (or the lack or mixing of colors in some systems) identifies which nucleotide is incorporated in each cluster. The dye then can be cleaved and the 3′-OH can be regenerated with a reducing agent such as tris-(2-carboxyethyl) phosphine. The cycle of nucleotide addition, elongation and cleavage can then begin again. Some DNA sequencers perform an internal quality filtering procedure called chastity filter, and reads that pass this filter are called PF for pass-filter. Chastity is defined as the ratio of the brightest base intensity divided by the sum of the brightest and second brightest base intensities. Clusters of reads can pass the filter if no more than 1 base call has a chastity value below 0.6 in the first 25 cycles. This filtration process can remove the least reliable clusters from the image analysis results.

Some platforms using patterned nano-well flow cells can give rise to approximately 70% PF reads. Such a relatively non-optimal PF reads can be due to the combination of multiple factors, including rapid exclusion amplification chemistry, inadequate adaptors in DNA fragment, non-optimal DNA concentration loaded per flow cell lane, non-optimal DNA seeding into each nano-well (e.g., empty or more than 1 fragment), and uncontrolled DNA spreading into adjacent wells during cluster generation step. An array of DNA binding regions separated by DNA non-binding regions (e.g., as described in various embodiments herein) may enable better DNA seeding with minimal DNA spreading and/or background signal, thereby improving DNA sequencing efficiency and quality.

The sequencing-by-ligation approach can use a patterned surface chemistry flow cell, wherein a single DNA nanoball is immobilized onto a specific patch or island coated with DNA binding chemistry. Here the cluster can be generated using solution-based rolling circle amplification (also called DNA nanoball generation). Specifically, DNA can be fragmented and ligated to the first of four adapter sequences. The template can be amplified, circularized and cleaved with a type II endonuclease. A second set of adapters can be added, followed by amplification, circularization and cleavage. This process can be repeated for the remaining two adapters. The final product can be a circular template with four adapters, each separated by a template sequence. Library molecules can undergo a rolling circle amplification step, generating a large mass of concatamers called DNA nanoballs, which then can be deposited onto the surface of a flow cell.

After DNA nanoball deposition, an anchor complementary to one of four adapter sequences and a fluorophore-labeled probe can be bound to each nanoball. The probe can be degenerate at all but the first position. The anchor and probe then can be ligated into position and imaged to identify the first base on either the 3′ or the 5′ side of the anchor. Next, the probe-anchor complex can be removed and the process can begin again with the same anchor but a different probe with the known base at the n+1 position. This can be repeated until five bases from the 3′ end of the anchor and five bases from the 5′ end of the anchor are identified. Another round of hybridization can occur, this time using anchors with a five-base offset identifying an additional five bases on either side of the anchor. Finally, this whole process can be repeated for each of the remaining three adapter sequences in the nanoball, generating paired-end reads.

The DNA nanoball-based sequencing technique can have non-optimal read efficiency, due to combination of multiple factors, including non-uniform DNA nanoball size, inadequate adaptors, non-optimal DNA nanoball concentration loaded per flow cell lane, and non-optimal DNA nanoball seeding into each DNA binding region (e.g., empty or more than 1 nanoballs, or detachment from the surface during the washing step of a specific cycle). An array of DNA binding regions separated by DNA non-binding regions (e.g., as described in various embodiments herein) may enable better DNA seeding with minimal DNA relocation and/or background signal, thereby improving DNA sequencing efficiency and quality.

Regardless of technologies used, NGS platforms can share similar workflow (except for single-molecule-based gene sequencing techniques)—including sample preparation, cluster generation, sequencing, and data analysis. However, Applicants note that NGS technologies can be divergent in flow cell design and operational principles. For optical detection-based NGS technologies, flow cells can contain either non-patterned or patterned surfaces for DNA capture and sequencing. Compared to non-patterned surfaces, patterned surfaces can have advantages associated with more efficient use of the flow cell surface area, yielding higher sequencing reads and data output. For example, patterned flow cells can be configured as patterned nano-well flow cells or patterned surface chemistry flow cells.

Further, embodiments also disclose devices and methods for making an array of molecules or nucleic acids attached to a surface of the device. Particular embodiments exploit the selective surface chemistry patterning to immobilize DNA molecules selectively onto specific patches or islands or regions of the substrate surface. Patterning of metal oxide films on the substrate surface can be achieved using the combination of photo lithography and metal oxide deposition. For example, either lift-off or etch method can be used to generate different patterns of metal oxide film on the substrate surface, depending on specific configuration of metal oxide patterns that are targeted.

Embodiments are exemplified and described herein with reference to metal oxide regions and non-metal oxide regions that are located on a substrate surface. The non-metal oxide region is the bare substrate region. However, the embodiments are not intended to be limited to those in which the regions are on a surface. Rather, in particular embodiments, a metal oxide region, bare substrate region or both can occur in a solid support or under the surface of the solid support (e.g., nano-well within the solid support or substrate). Furthermore, the location of a metal oxide region, base substrate region or both can change with respect to a solid support.

The change can be brought about by modifying the solid support, for example, by etching, reactive ion etching or polishing the solid support to bring a region to the surface from below. The change can be brought about by covering a region on a surface, by coating the surface or by building up the solid support.

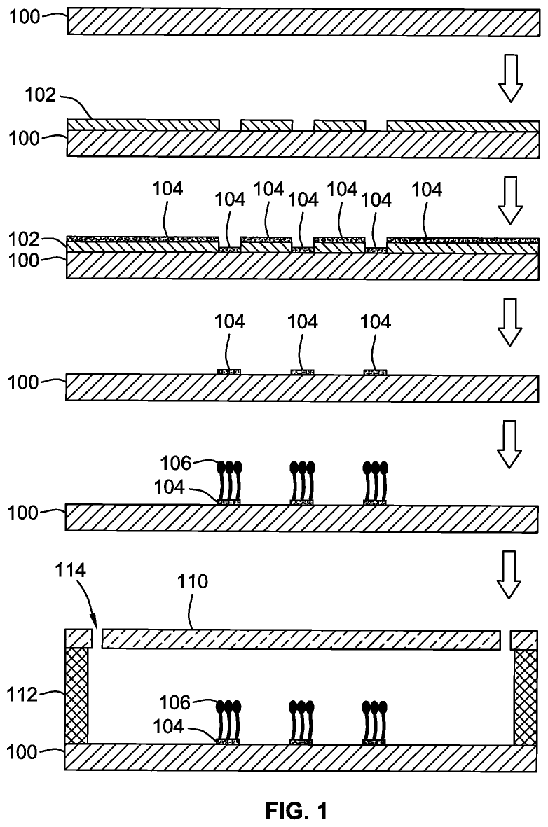

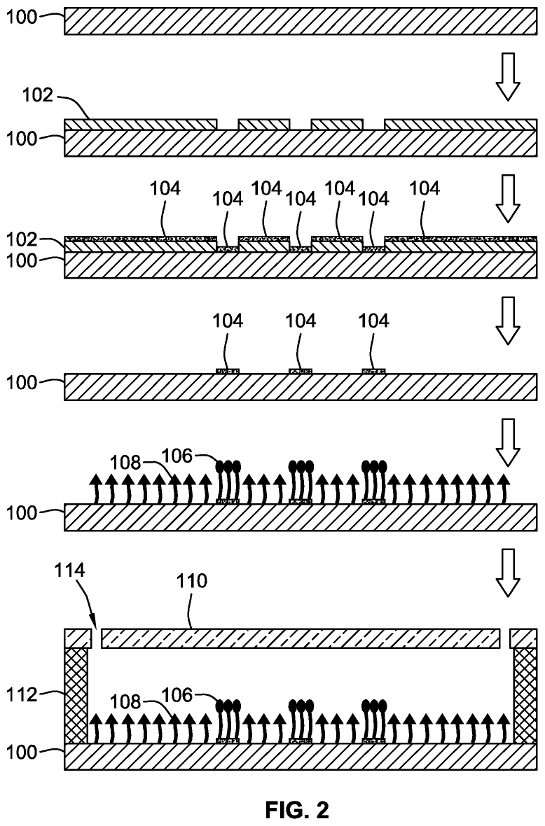

FIGS. 1 and 2 are schematic illustrations of the steps used in exemplary methods for selective coating of a substrate surface to generate a patterned surface comprising, consisting essentially of, or consisting of an array of metal oxide nano-patches with DNA-binding chemistry. In FIG. 1, a substrate 100 is first coated with a metal oxide 104 using a combination of lift-off photolithography and metal oxide deposition to form a patterned array of metal oxide nano-patches, followed by solution-based coating with an organophosphate 106 (or silane in an alternate embodiment) to form DNA-binding regions on the metal oxide nano-patches, and optionally further solution or vapor-based coating with a silane 108 (or organophosphate in an alternate embodiment) to form regions resistant to binding with DNA, proteins, and/or polynucleotides. The DNA-resistant regions can be located in the interstitial spaces between the metal oxide nano-patches. It should be noted that, in alternate embodiments, the nano-patches can be an array of silicon dioxide (SiO₂) or silicate nano-patches formed on a metal-oxide-coated substrate. Such embodiments would be similar in design to that disclosed in FIGS. 1 and 2, and these alternate embodiments could also incorporate the organophosphate and/or silane coatings referenced above and described in more detail below.

In some embodiments, photolithographic processes involve the deposition of a photoresist 102 onto the substrate 100. A pattern can be created in the photoresist 102 (e.g., using UV light). The photoresist can be selectively removed from exposed portions of the substrate 100. A metal oxide 104 can be deposited onto the photoresist and onto the exposed portions of the substrate 100. Afterwards, the metal oxide layer over the photoresist region can be lifted off, so an array of metal oxide nano-patches can be formed on the substrate surface. For example, a portion of the metal oxide layer disposed on the remaining photoresist (e.g., a remaining portion of the photoresist disposed on the substrate after patterning) can be removed (e.g., during the photoresist lift off), thereby leaving the array of metal oxide nano-patches disposed on the substrate surface.

The substrate 100 can be (e.g., comprise) glass, quartz, silicon, thermal plastic, or another suitable substrate material. The composition and geometry of a substrate can vary depending on the intended use and preferences of the user. The metal oxide 104 can be (e.g., comprise) Al₂O₃, ZnO₂, Ta₂O₅, Nb₂O₅, SnO₂, MgO, indium tin oxide, indium zinc oxide, CeO₂, CoO, Co₃O₄, Cr₂O₃, Fe₂O₃, Fe₃O₄, In₂O₃, Mn₂O₃, NiO, a-TiO₂ (anatase), r-TiO₂ (rutile), WO₃, Y₂O₃, ZrO₂, other metal oxides, or combinations thereof. In some embodiments, the metal oxide is transparent within visible wavelengths (e.g., from 400 nm to 700 nm). For example, the metal oxide can have a transmission within visible wavelengths of 50%, 60%, 70%, 80%, 90%, 95%, 99%, 100% or any ranges defined by the listed values.

In some embodiments, the metal oxide 104 is coated with an organophosphate layer 106 (e.g., to increase the ability of the metal oxide 104 to bind with DNA, proteins, and/or polynucleotides). For example, the organophosphate layer 106 is (e.g., comprises) one or more of amine-terminated organophosphate, epoxy-containing organophosphate, or carboxylate organophosphate. Following the self-assembly monolayer formation of organophosphate 106 on the metal oxide 104 surface, the amine groups can be used to either electrostatically attract DNA for immobilization, or to covalently attach DNA via a bifunctional linker (e.g., BS3, or anhydride polymer), where the epoxy groups can be used to covalently attach amine-terminated DNA. The carboxylate group of carboxylate organophosphate can be further converted into anhydride or N-hydroxysuccinimide ester for covalent bonding formation with amine-terminated DNA.

In the embodiments shown in FIG. 2, a silane layer 108 is provided in the interstitial spaces between the metal oxide nano-patches 104. The silane 108 may be (e.g., comprise) a polyethylene glycol-containing silane. For example, the self-assembled monolayer of polyethylene glycol-containing silane formed on the interstitial area can make these areas resistant to DNA binding or immobilization, thereby preventing DNA-binding to these regions or preventing DNA spreading during cluster generation step of a sequencing nm.

The substrate 100 surface is intended to mean an external part or external layer of a solid support. The metal oxide region or patch 104 is intended to mean an area in a substrate or on a surface that contains a metal oxide film. The metal oxide region 104 can have a continuous coating of one or more type of metal oxide. The metal oxide region 104 can have a composition and thickness that is stable throughout the assays. The thickness of the metal oxide film 104 can be at least about 1 nanometer (nm). For example, the metal oxide film 104 is 10 nm, 50 nm, 100 nm, 500 nm, 5 micrometers (μm), or any range defined by any of the listed values.

The interstitial region is intended to mean an area in the substrate 100 or on a surface that separates or lies between DNA-binding regions or spots or patches (e.g., metal oxide regions or patches 104). The geometric shape and size of a DNA binding region on a surface can be varied, depending on applications. The shape can be, without limitation, rectangular, square, circular, elliptical, oval, triangular, polygonal, trapezoidal or irregular. The size can be a size that accommodates only a single nucleic acid molecule, or a single DNA nanoball, or a colony formed via bridge amplification, or a colony formed via exclusion amplification, or a colony formed via template walking. The bridge amplification can be primed by primer nucleic acids that are attached to a gel layer that is in contact with a DNA binding region.

When processing of the substrate 100 is finished, the substrate 100 may be protected with a glass cover 110 to form a microfluidic flow cell device. The glass cover 110 may be supported and bonded to the substrate 100 using a bonding material 112. The bonding material could be tape, a metal or metal oxide bonded to both the substrate 100 and glass cover 110, a glass material, or another suitable material. The microfluidic channel(s) of the flow cell device can be defined, for example, by the openings of the tape used, or by pre-etched channel(s) within the glass cover 100. Depending on the specific configuration of the flow cell device, either the glass cover 110 or the substrate 100 may include openings 114 for the introduction of biological samples and other chemicals.

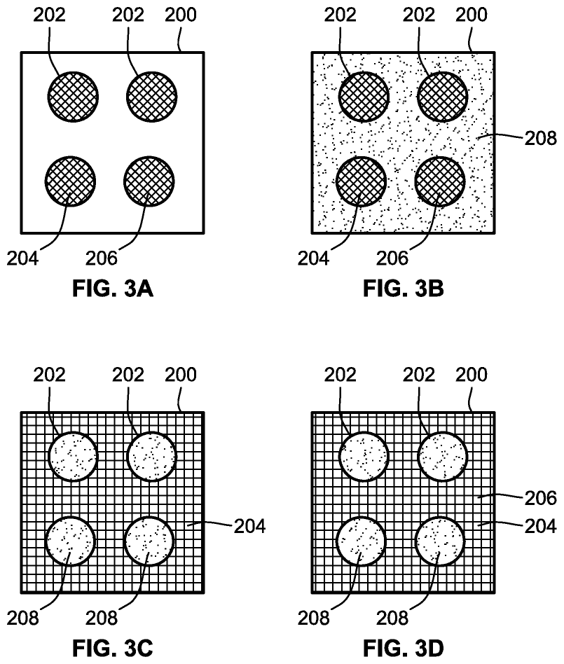

FIGS. 3A, 3B, 3C, and 3D show four alternate embodiments. In FIG. 3A, the organophosphate 206 is used to coat the metal oxide regions 204 (e.g., patches or islands). In FIG. 3B, the organophosphate 206 is used to coat the metal oxide regions 204 (e.g., patches or islands), while silane 208 can then be used to coat the SiO₂ or silicate regions (e.g., interstitial regions) when the substrate is a glass, silicon, or a pure silica. In FIG. 3C, the silane 208 is used to coat the SiO₂ or silicate regions (e.g., patches or islands) when the substrate is a glass, silicon, or a pure silica. In FIG. 3D, the organophosphate 206 is used to coat the metal oxide regions 204 (e.g., interstitial regions), while the silane 208 is then used to coat the SiO₂ or silicate regions (e.g., patches or islands) when the substrate is a glass, silicon, or a pure silica. When the substrate is plastic, silane can be replaced with respective hydrogel polymers such as acrylate polymers. The embodiment of FIG. 3B is consistent with the embodiment described above for FIG. 2.

The array of regions can appear as a grid of spots or patches 202 on a substrate 200, and/or can form a repeating pattern (e.g., hexagonal, rectilinear, grid patterns) or an irregular, non-repeating pattern. The pitch can be the same between different pairs of nearest neighbors or can be varied between different pairs of nearest neighbors. A metal oxide layer 204 can be deposited on a surface using methods known in the art such as wet plasma etching, dry plasma etching, atomic layer deposition, ion beam etching, e-beam evaporation, chemical vapor deposition, sputtering or the like using commercially available systems.

In some embodiments configured as shown in FIGS. 3C and 3D, the substrate 200 is first coated with a layer of metal oxide 204, followed by photolithography and reactive ion etching to remove portions of the metal oxide layer in the patterned, exposed regions, thereby forming an array of SiO₂ or silicate nano-patches separated by continuous metal oxide film 204. Afterwards, the substrate can be coated with an organophosphate 206 to form DNA non-binding region on the metal oxide regions 204, and further solution or vapor-based coating with a silane 208 to form DNA binding regions on the array of spots 202. The organophosphate can be one of a polyethylene glycol-containing organophosphate and/or polyvinyl phosphoric acid. The silane can be one of amine-terminated silane, epoxy-containing silane, maleimide polyethylene-glycol-containing silane, and/or carboxylate silane.

The substrate 200 can be glass, quartz, silicon, thermal plastic, or another suitable material. The metal oxide 204 can be Al₂O₃, ZnO₂,Ta₂O₅, Nb₂O₅, SnO₂, MgO, indium tin oxide, indium zinc oxide, CeO₂, CoO, Co₃O₄, Cr₂O₃, Fe₂O₃, Fe₃O₄, In₂O₃, Mn₂O₃, NiO, a-TiO₂ (anatase), r-TiO₂ (rutile), WO₃, Y₂O₃, ZrO₂, other metal oxides, or combinations thereof. For example, the metal oxide 204 is transparent within visible wavelengths (e.g., from 400 to 700 nm). The organophosphate layer 206 can be polyethylene glycol (PEG)-containing organophosphate or poly(vinyl phosphoric acid). The self-assembled monolayer of these organophosphate 206 formed on the interstitial metal oxide areas 204 can make these areas resistant to DNA-binding or immobilization, thereby preventing binding to DNA, proteins, and/or polynucleotides in these regions or preventing the DNA, proteins, and/or polynucleotides from spreading during cluster generation step of a typical sequencing run.

In some embodiments, the silane 208 is one of amine-terminated silane, epoxy-containing silane, carboxylate silane, and/or maleimide PEG silane. Following the self-assembly monolayer formation of silane 208 on the substrate surface, the amine groups can be used to either electrostatically attract DNA for immobilization or to covalently attach DNA via a bifunctional linker (e.g., BS3 or an anhydride polymer), where the epoxy groups can be used for covalent bonding formation with amine-terminated DNA, the maleimide groups can be used for covalent bonding formation with thiol-terminated DNA. The carboxylate silane can be further converted into anhydride for covalent bonding formation with amine-terminated DNA.

Organophosphoric acid molecules can form stable P-O-M bond with metal oxide, but not silicon oxide, in aqueous solution. However, silane can form self-assembled monolayer on both metal oxide and silicon oxide surface. Thus, it can be beneficial to coat the array of metal oxide regions with organophosphate first, followed by silane coating of the bare substrate regions. The silane coating can be done in solution-based or vapor deposition approach.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosed embodiments. No language in the specification should be construed as indicating any non-claimed element as essential.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A patterned flow cell comprising:
a substrate comprising a patterned array of metal oxide nano-patches, each of the metal oxide nano-patches comprising an organophosphate coating layer to increase the ability of the metal oxide to bind with at least one of DNA, proteins, or polynucleotides; and
a silane coating layer on interstitial spaces between the metal oxide nano-patches to prevent the binding of at least one of polynucleotides, DNA, or proteins in the interstitial spaces.

2. The patterned flow cell of claim 1, wherein the metal oxide nano-patches comprise at least one of Al₂O₃, ZnO₂,Ta₂O₅, Nb₂O₅, SnO₂, In₂o₃, a-TiO₂, r-TiO₂, indium tin oxide, indium zinc oxide, or ZrO₂.

3. The patterned flow cell of claim 1, wherein the metal oxide nano-patches comprise at least one of MgO, NiO, CeO₂, CoO, Co₃O₄, CrO₃, Fe₂O₃, Fe₃O₄, WO₃, Y₂O₃, or Mn₂O₃.

4. The patterned flow cell of claim 1, wherein the organophosphate coating layer comprises at least one of amine-terminated organophosphate, epoxy-containing organophosphate, or carboxylate organophosphate.

5. The patterned flow cell of claim 1, wherein the silane coating layer comprises polyethylene-glycol-containing silane.

6. The patterned flow cell of claim 1, wherein a shape of the metal oxide nano-patches is one of rectangular, circular, elliptical, oval, triangular, or trapezoidal.

7. The patterned flow cell of claim 1, wherein the substrate comprises at least one of glass, quartz, silicon, or plastic.

8. The patterned flow cell of claim 1, wherein a thickness of the metal oxide nano-patches is in a range from one nanometer to five micrometers.

9. The patterned flow cell of claim 1, wherein the metal oxide nano-patches are transparent to light with wavelengths in a range from 400 nanometers to 700 nanometers.

10. A patterned flow cell comprising:
a substrate comprising a patterned array of SiO₂ or silicate nano-patches, each of the SiO₂ or silicate nano-patches comprising a silane coating layer to increase the ability of the SiO₂ or silicate nano-patch to bind with at least one of DNA, proteins, or polynucleotides; and
a metal oxide layer disposed on interstitial spaces between the SiO₂ or silicate nano-patches, and an organophosphate coating layer on the metal oxide layer to prevent the binding of at least one of polynucleotides, DNA, or proteins in the interstitial spaces.

11. The patterned flow cell of claim 10, wherein the metal oxide layer comprises at least one of Al₂O₃, ZnO₂,Ta₂O₅, Nb₂O₅, SnO₂, In₂O₃, a-TiO₂, r-TiO₂, indium tin oxide, indium zinc oxide, or ZrO₂.

12. The patterned flow cell of claim 10, wherein the metal oxide layer comprises at least one of CeO₂, CPO, Co₃O₄, CrO₃, Fe₂O₃, Fe₃O₄, NiO, WO₃, Y₂O₃, MgO, or Mn₂O₃.

13. The patterned flow cell of claim 10, wherein the organophosphate coating layer comprises at least one of a polyethylene glycol-containing organophosphate or polyvinyl phosphoric acid.

14. The patterned flow cell of claim 10, wherein the silane coating layer comprises at least one of amine-terminated silane, epoxy-containing silane, maleimide polyethylene-glycol-containing silane, or carboxylate silane.

15. The patterned flow cell of claim 10, wherein the substrate comprises at least one of glass, quartz, silicon, or plastic.

16. The patterned flow cell of claim 10, wherein a thickness of the SiO₂ or silicate nano-patches is in a range from one nanometer to five micrometers.

17. A method of manufacturing a patterned flow cell comprising the steps of:
depositing an array of metal oxide nano-patches onto a substrate;
coating the metal oxide nano-patches with a first material which increases the ability of the metal oxide to bind with at least one of DNA, proteins, or polynucleotides;
depositing a coating layer of a second material on the substrate in interstitial spaces between the nano-patches to prevent the binding of at least one of polynucleotides, DNA, or proteins in the interstitial spaces.

18. The method of claim 17, wherein depositing the array of metal oxide nano-patches comprises depositing the array of metal oxide nano-patches using a combination of photolithography and metal-oxide deposition.

19. The method of claim 17, wherein the first material comprises at least one of amine-terminated organophosphate, epoxy-containing organophosphate, or carboxylate organophosphate.

20. The method of claim 17, wherein the second material comprises a polyethylene-glycol-containing silane.

21-24. (canceled)
